(19) United States
(12) Patent Application Publication
(10) Pub. No.: US 2022/0265876 A1
(43) Pub. Date: Aug. 25, 2022

(54) SANITATION DEVICE

(71) Applicant: Tekna, Inc., Kalamazoo, MI (US)

(72) Inventors: Adam Wayne Dudycha, Paw Paw, MI (US); Kyle Stephen Spieker, Kalamazoo, MI (US); Joseph Richard Regimbald, Kalamazoo, MI (US); Michael Rex Gruizinga, Kalamazoo, MI (US); Joseph Quentin Marietta, Kalamazoo, MI (US); Timothy James LaRoy, Kalamazoo, MI (US); Aaron Michael Betker, Kalamazoo, MI (US); Robert Aaron Feeman, Kalamazoo, MI (US); Benjamin John Purrenhage, Kalamazoo, MI (US); Lars Robert Chrisman, Gobles, MI (US)

(73) Assignee: Tekna, Inc., Kalamazoo, MI (US)

(21) Appl. No.: 17/548,955

(22) Filed: Dec. 13, 2021

Related U.S. Application Data
(60) Provisional application No. 63/153,527, filed on Feb. 25, 2021.

(51) Int. Cl. A61L 2/10; A61L 2/24

(52) U.S. Cl. CPC A61L 2/10; A61L 2/24; A61L 2202/11; A61L 2202/121; A61L 2202/122; A61L 2202/14; A61L 2202/15; A61L 2202/16

(57) ABSTRACT

A sanitation device is disclosed. The sanitation device includes a housing, one or more reflective panels, one or more bulb assemblies, and an access door. The one or more reflective panels and the access door define a chamber. The one or more bulb assemblies are positioned within the chamber.

### CROSS-REFERENCE TO RELATED APPLICATION

This application claims priority to and the benefit under 35 U.S.C. § 119(e) of U.S. Provisional Patent Application No. 63/153,527, filed on Feb. 25, 2021, entitled SANITATION DEVICE, the entire disclosure of which is hereby incorporated herein by reference.

### FIELD OF THE DISCLOSURE

The present disclosure generally relates to a sanitation device. More specifically, the present disclosure relates to a sanitation device with an ultraviolet operating wavelength.

### BACKGROUND

A variety of microorganisms are undesirable to consumers. Some of these microorganism can lead to infectious diseases or general discomfort among humans. Additional solutions are needed that provide consumers with the ability to cleanse or sanitize aspects of the environment with which they interact.

### SUMMARY

According to a first aspect of the present disclosure, a sanitation device includes a housing and an access door. The access door is coupled to the housing. The access door is movable between an open position and a closed position. The access door includes an interior surface. A plurality of reflective panels are positioned within the housing. One of the plurality of reflective panels is coupled to the interior surface of the access door. A cavity is defined by the housing and at least one of the plurality of reflective panels. A chamber is defined by the plurality of reflective panels and the access door. A shelf is positioned within the chamber and configured to receive an article. One or more bulb assemblies are positioned within the chamber. The one or more bulb assemblies each includes a bulb and a receptacle. The bulb is operable between an energized state and a de-energized state. The bulb emits light with a wavelength in the range of about 100 nm to about 400 nm when in the energized state. Each of the plurality of reflective panels is configured to reflect the light emitted by the bulb.

According to a second aspect of the present disclosure, a method of operating a sanitation device includes the steps of determining an access door is in a closed position; supplying power to a bulb of a bulb assembly; monitoring an electrical circuit that comprises the bulb assembly; determining whether the bulb of the bulb assembly is in an energized state or a de-energized state based upon the monitoring of the electrical circuit that comprises the bulb assembly; and sensing a temperature within a chamber with a temperature sensor.

These and other aspects, objects, and features of the present disclosure will be understood and appreciated by those skilled in the art upon studying the following specification, claims, and appended drawings.

### BRIEF DESCRIPTION OF THE DRAWINGS

In the drawings:

FIG. 1 is a front perspective view of a sanitation device, illustrating articles placed on a shelf thereof, according to one example;

FIG. 2 is a rear perspective view of FIG. 1 of the sanitation device, illustrating an anchor point, according to one example;

FIG. 3 is a bottom view of the sanitation device, illustrating vents, according to one example;

FIG. 4 is a cross-sectional view, taken along line IV-IV of FIG. 1, illustrating bulb assemblies within a chamber of the sanitation device, according to one example;

FIG. 5 is a cross-sectional view, taken along line V-V of FIG. 1, illustrating the bulb assemblies within the chamber of the sanitation device, according to one example;

FIG. 6 is a front perspective view of a sanitation device, illustrating articles placed on the shelf thereof, according to another example;

FIG. 7 is a rear perspective view of the sanitation device of FIG. 6, illustrating a user interface on an access door thereof;

FIG. 8 is a cross-sectional view of the sanitation device, taken along line VIII-VIII of FIG. 6, illustrating the bulb assemblies within the chamber of the sanitation device;

FIG. 9 is a cross-sectional view of the sanitation device, taken along line IX-IX of FIG. 6, illustrating the bulb assemblies within the chamber of the sanitation device;

FIG. 10 is a front perspective view of a sanitation device, illustrating articles placed on the shelves thereof, according to one example;

FIG. 11 is a rear perspective view of the sanitation device of FIG. 10, illustrating a cord-keeping structure and a grab handle, according to one example;

FIG. 12 is a cross-sectional view of the sanitation device, taken along line XII-XII of FIG. 10, illustrating various components of the sanitation device;

FIG. 13 is a cross-sectional view of the sanitation device, taken along line XIII-XIII of FIG. 10, illustrating the various components of the sanitation device;

FIG. 14 is a schematic representation of the sanitation device, illustrating a controller, according to one example; and

FIG. 15 is a flow diagram, illustrating a method of operating the sanitation device, according to one example.

### DETAILED DESCRIPTION

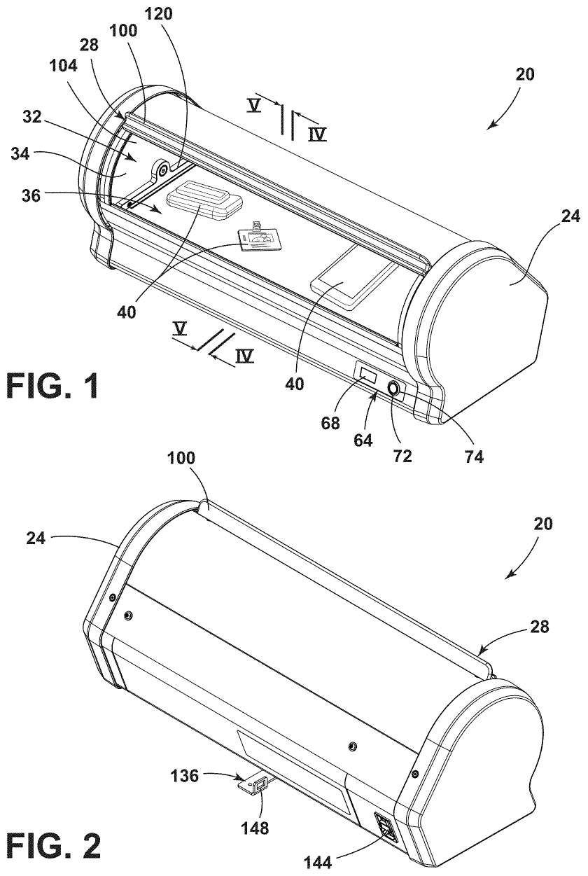

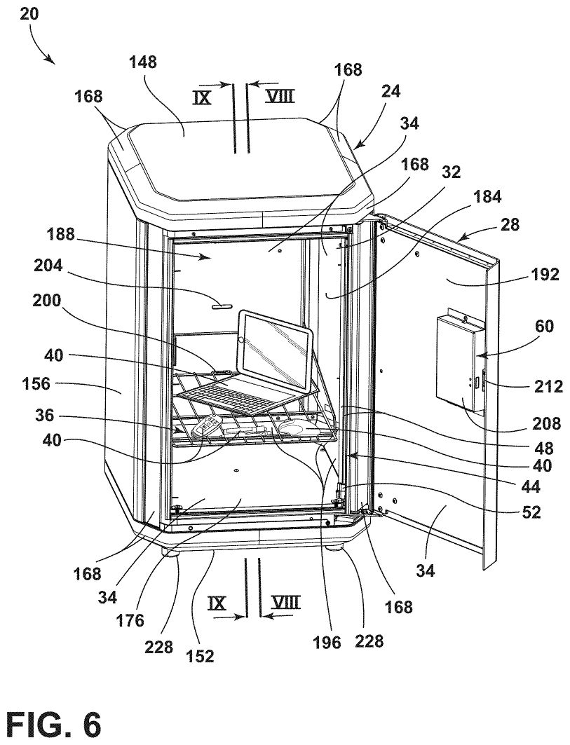

For purposes of description herein, the terms “upper,” “lower,” “right,” “left,” “rear,” “front,” “vertical,” “horizontal,” and derivatives thereof shall relate to the concepts as oriented in FIGS. 1, 6, and/or 10. However, it is to be understood that the concepts may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The present illustrated embodiments reside primarily in combinations of method steps and apparatus components related to a sanitation device. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

As used herein, the term “and/or,” when used in a list of two or more items, means that any one of the listed items can be employed by itself, or any combination of two or more of the listed items, can be employed. For example, if a composition is described as containing components A, B, and/or C, the composition can contain A alone; B alone; C alone; A and B in combination; A and C in combination; B and C in combination; or A, B, and C in combination.

In this document, relational terms, such as first and second, top and bottom, and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

As used herein the terms “the,” “a,” or “an,” mean “at least one,” and should not be limited to “only one” unless explicitly indicated to the contrary. Thus, for example, reference to “a component” includes embodiments having two or more such components unless the context clearly indicates otherwise.

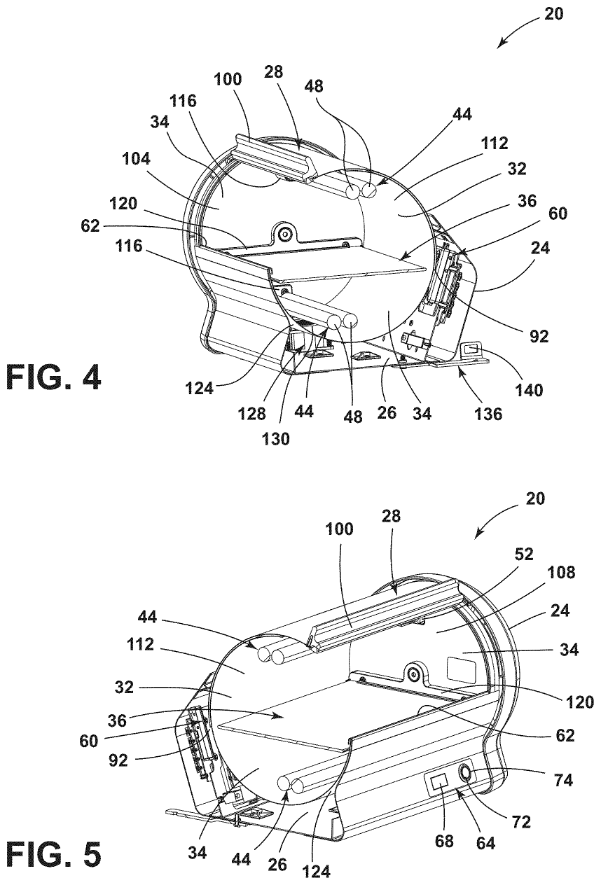

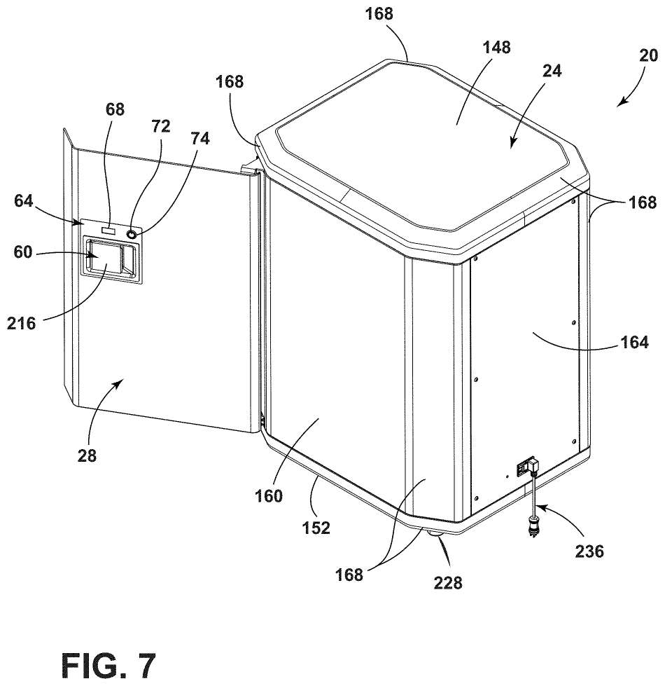

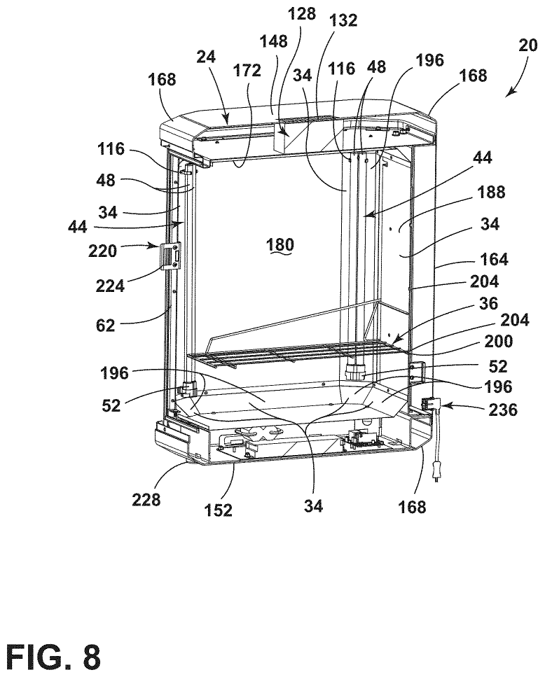

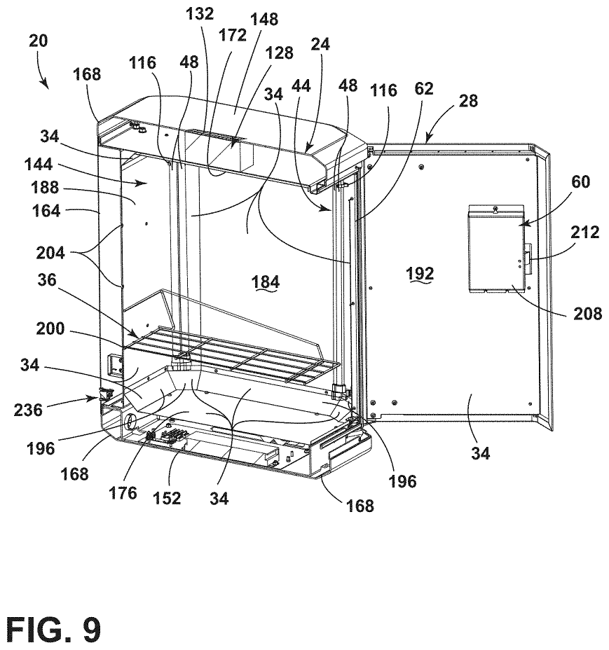

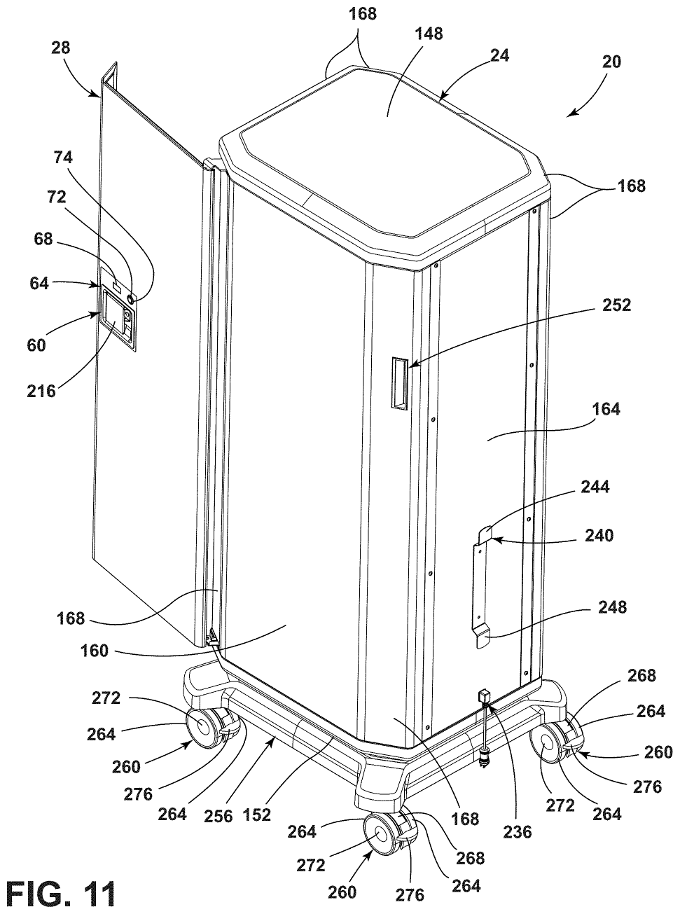

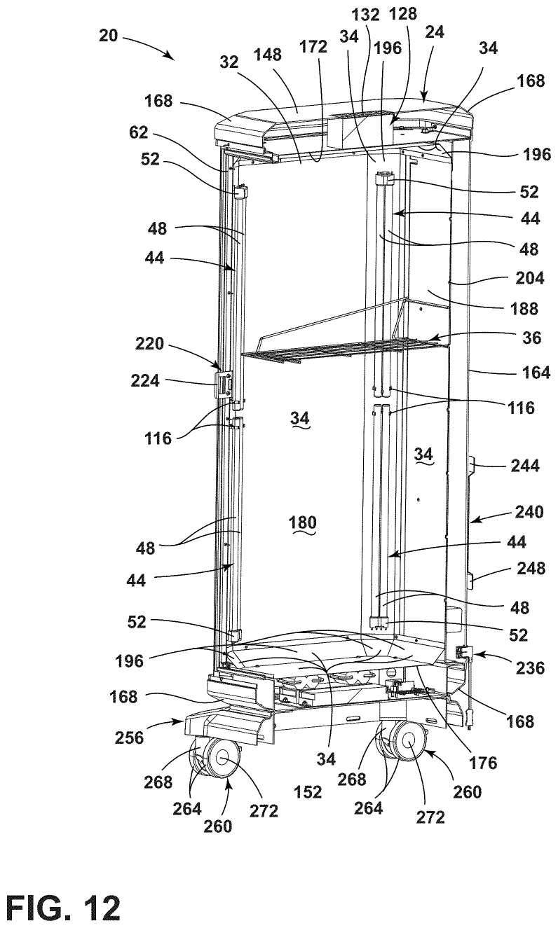

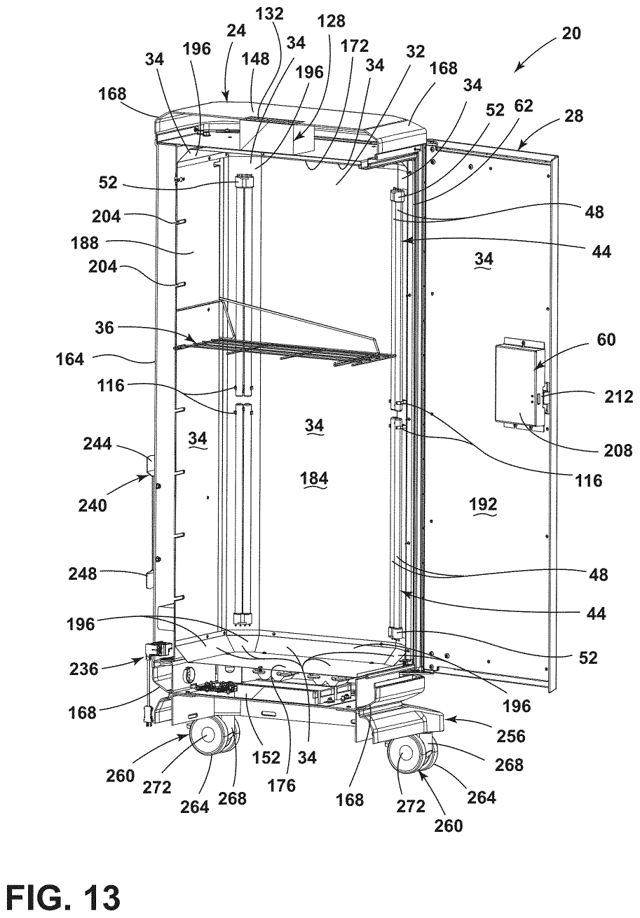

Referring to FIGS. 1-14, reference numeral 20 generally designates a sanitation device. The sanitation device 20 includes a housing 24, a cavity 26 defined by the housing 24, an access door 28, and a chamber 32. The access door 28 is coupled to the housing 24 and is movable between an open position and a closed position. One or more reflective panels 34 are positioned within the cavity 26. The chamber 32 is defined by the one or more reflective panels 34. In some examples, the chamber 32 may also be defined by the access door 28. An interior surface of the access door 28 includes one of the reflective panels 34. A shelf 36 is positioned within the chamber 32 and is configured to receive an article 40. A bulb assembly 44 is positioned within the chamber 32. The bulb assembly 44 includes a bulb 48 and a receptacle 52. The bulb 48 is operable between an energized state and a de-energized state. In various examples, the bulb 48 is configured to emit light with a wavelength in the range of about 100 nm to about 400 nm. For example, the light emitted by the bulb 48 may be ultraviolet light. In one specific example, the wavelength of light emitted by the bulb 48 can be in the range of about 200 nm to about 280 nm (e.g., UVC light). The one or more reflective panels 34 are configured to reflect the light emitted by the bulb 48. In various examples, the reflective panels 34 can be provided with a reflective coating that results in at least 90% of the light from the bulbs 48 being reflected. Said another way, the reflective panels 34 reflect at least 90% of incident light at the wavelength(s) of operation of the bulbs 48. For example, the reflective panels 34 can reflect at least 90% of incident light, at least 95% of incident light, at least 98% of incident light, 100% of incident light, and/or combinations or ranges thereof

Referring again to FIGS. 1-14, a lock assembly 60 is coupled to the access door 28 and is operable between an engaged state and a disengaged state. The lock assembly 60 can prevent the access door 28 from being moved from the closed position to the open position when the lock assembly 60 is in the engaged state. When the access door 28 is in the open position, an opening 62 of the sanitation device 20 is revealed. The opening 62 is utilized by a user to access the chamber 32. A user interface 64 is provided. The user interface 64 includes a display screen 68 and a button 72. In some examples, the user interface 64 can be utilized by a user to select a cleansing cycle based upon a duration of the cleansing cycle and/or a target pathogen or microorganism to be cleansed from the article 40. In various examples, the button 72 can be provided with a perimeter structure 74 that is positioned about a perimeter or circumference of the button 72. The perimeter structure 74 can be employed (e.g., illuminated) to convey a status and/or notification for the sanitation device 20. The sanitation device 20 includes a controller 76. The controller 76 includes a processor 80 and memory 84. The memory 84 includes software routines 88 stored therein that can be executed by the processor 80. The chamber 32 is defined by the housing 24 and the access door 28.

Referring again to FIGS. 1-5, the access door 28 is depicted in the open position. When the access door 28 is in the open position, a rearward edge 92 of the access door 28 can be actuated or moved into the cavity 26 of the housing 24. The cavity 26 can be defined by an interior surface of the housing 24 and an exterior surface of the one or more reflective panels 34. Movement of the access door 28 between the open position and the closed position can be accomplished by a user applying a force to a handle 100 of the access door 28. With the access door 28 in the open position, a user may place the article 40 within the chamber 32 by resting the article 40 upon the shelf 36. Once the user has placed the article 40 upon the shelf 36 and wishes to begin a cleansing cycle, the user may actuate the access door 28 to the closed position. In some examples, the user may manually actuate the access door 28 to the closed position by applying a force to the handle 100 (e.g., a downward force). Alternatively, the access door 28 may be actuated between the open position and the closed position by activation of a motor coupled to the access door 28. In such an example, the user can initiate a closing sequence of the access door 28 by activating the motor coupled to the access door 28. For example, the closing sequence may be initiated by the user pressing the button 72 while the access door 28 is in the open position. Regardless of the source of the force that results in placing the access door 28 in the closed position, the lock assembly 60 may be engaged once the access door 28 is in the closed position. In some examples, the lock assembly 60 can remain in the disengaged state while the access door 28 is in the closed position until such time as the user initiates a cleansing cycle of the sanitation device 20. Once the access door 28 is placed in the closed position and the lock assembly 60 is placed in the engaged state, the cleansing cycle may be initiated as a result of the actuation of the button 72. In the depicted example, the lock assembly 60 is positioned between the first and second ends 104, 108 within the cavity 26 and proximate to a rear of the sanitation device 20. The lock assembly 60 can engage with the access door 28 when the access door 28 is in the closed position to prevent actuation of the access door 28 to the open position. For example, the lock assembly 60 can engage with a structure along the rearward edge 92 to retain the access door 28 in the closed position.

Referring further to FIGS. 1-5, upon initiation of the cleansing cycle, the bulb assemblies 44 can enter into the energized state. In the energized state, the bulbs 48 of the bulb assemblies 44 receive electrical energy from a power source (e.g., a wall outlet), which is converted into light that is emitted by the bulbs 48. In various examples, the light emitted by the bulbs 48 can have a wavelength that corresponds with microorganism disinfection of the article 40. For example, the wavelength emitted by the bulbs 48 can induce cell death of microorganisms, such as bacteria, fungi, viruses, and so on. In one specific example, the wavelength emitted by the bulbs 48 may be 254 nm. The reflective panels 34 are arranged within the chamber 32 such that the chamber 32 is awash with light from the bulbs 48. Accordingly, the arrangement of the reflective panels 34 can prevent dead spots or voids in light coverage. In the depicted example, the chamber 32 is generally cylindrical in shape with the reflective panels 34 that define the chamber 32 including a first end 104, a second end 108, and a central body 112 that extends between the first and second ends 104, 108. Accordingly, the geometry of the chamber 32 encourages reflection of the light emitted by the bulbs 48 toward the shelf 36. In various examples, the shelf 36 can be made of a material that has a high degree of transmission or is transparent at the wavelength or wavelengths of light emitted from the bulbs 48. Therefore, a surface of the article 40 that is in direct contact with the shelf 36 can be exposed to the light emitted from the bulbs 48 and thereby disinfected.

Referring still further to FIGS. 1-5, in the depicted example, the shelf 36 is coupled to the first and second ends 104, 108 and extends therebetween such that the shelf 36 is suspended between diametrically opposed bulb assemblies 44. Accordingly, light emitted from the bulbs 48 of a lower one of the bulb assemblies 44 may directly contact the surface of the article 40 that rests upon the shelf 36 prior to reflection off of one or more of the reflective panels 34. Similarly, light emitted from the bulbs 48 of an upper one of the bulb assemblies 44 may directly contact surfaces of the article 40 that are opposite to the surface of the article 40 that is in direct contact with the shelf 36. It is contemplated that a single one of the bulb assemblies 44 may be employed as the reflective properties of the reflective panels 34 and the transparent nature of the shelf 36 at the wavelength or wavelengths of light emitted from the bulbs 48 may be sufficient to disinfect the surfaces of the article 40. However, it may be beneficial to provide more than one of the bulb assemblies 44 as an overall time of the cleansing cycle may be decreased and/or an efficiency of the cleansing cycle may be increased by providing a plurality of the bulb assemblies 44. The bulb assemblies 44 can include retention clips 116 that engage with the bulbs 48 at an opposing end of the bulbs 48 to the positioning of the receptacle 52 such that the bulbs 48 are retained within a designed or desired position and the bulbs 48 are provided with additional support. For example, the receptacles 52 may be positioned on the second end 108 of the chamber 32 while the retention clips 116 may be positioned on the first end 104 of the chamber 32. The bulb assemblies 44 are coupled to the first and second ends 104, 108 of the chamber 32 and positioned radially inward from the central body 112 of the chamber 32 such that light emitted in any direction from the bulbs 48 contacts at least one of the reflective panels 34 and is directed toward the shelf 36. In various examples, the shelf 36 can be coupled to the first and second ends 104, 108 by support brackets 120 that receive the shelf 36 to enable suspending the shelf 36 between the bulb assemblies 44.

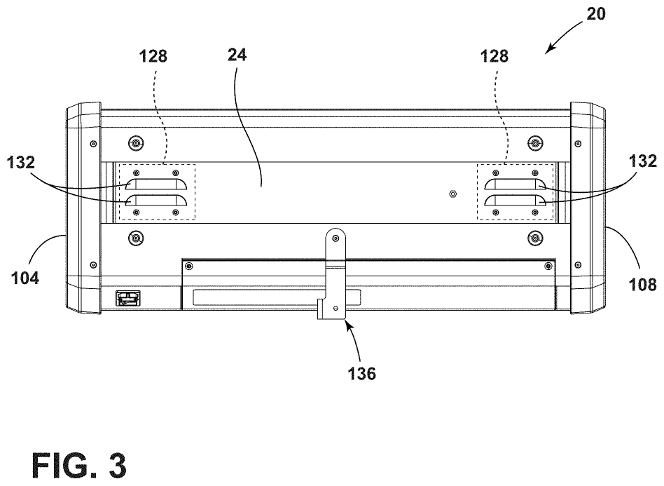

Referring again to FIGS. 1-5, the cavity 26 can be defined by the housing 24 and an exterior surface 124 of the central body 112. The cavity 26 can be utilized to house additional components of the sanitation device 20. In some examples, a circulation device 128 (e.g., a fan) can be provided and utilized to decrease an operating temperature of the sanitation device 20. In such examples, an orifice 130 may be defined by one or more of the reflective panels 34 at a region proximate to the circulation device 128. In various examples, if N number of cleansing cycles are executed within X period of time, the circulation device 128 may be activated to circulate air or another cooling medium through the sanitation device 20. In so doing, overheating of the sanitation device 20 can be prevented, a cool down time between cleansing cycles can be decreased, and/or an operating efficiency of the sanitation device 20 can be maintained. One or more vents 132 may be provided in an underside of the sanitation device 20 to encourage expulsion of heated air from the interior of the sanitation device 20 and/or provide an air intake for circulation of fresh air within the interior of the sanitation device 20. For example, the circulation device 128 may be positioned between the orifice 130 and one or more of the vents 132 such that fresh air may be introduced to the chamber 32 and/or heated air may be expelled from the chamber 32. The arrangement between the circulation device 128, the orifice 130, and the vents 132 may be duplicated proximate to the second end 108, as can be seen in FIG. 3. In various examples, the sanitation device 20 may be provided with an anchor point 136 that defines an anchor aperture 140. The anchor aperture 140 may be utilized to secure the sanitation device 20 to a fixed body (e.g., a wall, furniture, or other suitable support structure). Accordingly, the anchor point 136 may be utilized to prevent damage to the sanitation device 20 as a result of unintentional dislodging of the sanitation device 20 from a use location and/or prevent theft of the sanitation device 20. A power receptacle 144 may be provided on a rearward side of the sanitation device 20. The power receptacle 144 can receive power from the power source by way of a power cord (not shown). The exemplary sanitation device 20 depicted may be utilized for cleansing a variety of items, such as, but not limited to, mobile devices, wallets, keys, and the like. One of skill in the art will recognize that the identity of the article 40 to be cleansed is limited to the available physical space provided by the shelf 36 within the dimensions of the chamber 32. For example, the article 40 may occupy a significant portion of an available volume within the chamber 32. The available volume within the chamber 32 can be defined by distances between a top surface of the shelf 36 and each of the reflective panels 34. It is contemplated that a minimum distance between terminal edges or surfaces of the article 40 and each of the reflective panels 34 may be beneficial to maintain sufficient exposure to the light from the bulb(s) 48 and efficiently cleanse the article 40 during a cleansing cycle. The minimum distances between the terminal edges or surfaces of the article 40 and each of the reflective panels 34 can be dependent on the dimensions and/or shape of the chamber 32, the number of bulb assemblies 44 employed, a desired duration of time for the cleansing cycle, and/or a target pathogen or microorganism to be cleansed. In various examples, the minimum distance between the terminal edges or surfaces of the article 40 and each of the reflective panels 34 may be about 40%, about 35%, about 30%, about 25%, about 20%, about 15%, about 10%, or about 5% of the distance between the top surface of the shelf 36 and the given reflective panel 34,

Referring now to FIGS. 6-13, additional examples of the sanitation device 20 are shown. The depicted examples of the sanitation device 20 may provide a greater volume for the chamber 32. Accordingly, the depicted examples of the sanitation device 20 may be utilized for the cleansing of items or articles 40 that are larger than the available physical dimensions of the chamber 32 in the preceding figures. While the examples of the sanitation device 20 in the present figures may be utilized for articles 40 that are larger in size, this does not preclude the use of the present sanitation device 20 examples for smaller items, such as those that would fit in the chamber 32 of the sanitation device 20 depicted in FIGS. 1-5. In the depicted examples, the housing 24 defines the cavity 26 as generally rectangular in shape. Defining the generally rectangular shape of the cavity 26, the housing 24 is provided with a top wall 148, a bottom wall 152, a left wall 156, a right wall 160, and a rear wall 164. The access door 28 can act as a movable front wall of the housing 24. Adjacent edges of the top wall 148, the bottom wall 152, the left wall 156, the right wall 160, and/or the rear wall 164 can be coupled to one another by chamfered corners 168.

Referring again to FIGS. 6-13, angular relationships between the reflective panels 34 that define the chamber 32 generally correspond with the angular relationships between the various walls of the housing 24. More specifically, the reflective panels 34 can include a top panel 172, a bottom panel 176, a left panel 180, a right panel 184, a rear panel 188, a front panel 192, and junction panels 196. The top panel 172 corresponds with and is parallel to the top wall 148, the bottom panel 176 corresponds with and is parallel to the bottom wall 152, the left panel 180 corresponds with and is parallel to the left wall 156, the right panel 184 corresponds with and is parallel to the right wall 160, the rear panel 188 corresponds with and is parallel to the rear wall 164, the front panel 192 corresponds with and is parallel to a housing 24 portion of the access door 28, and the junction panels 196 correspond with and are parallel to the chamfered corners 168. In various examples, the chamfered corners 168 that are positioned at the junction between the top wall 148 and the access door 28, as well as the bottom wall 152 and the access door 28 may lack corresponding junction panels 196 in an effort to provide a larger area to the opening 62 that the user utilizes to access the chamber 32. In general, the junction panels 196 can be utilized to reduce a number of angles within the chamber 32 that are less than or equal to 90°. Therefore, edges of the junction panels 196 may form obtuse angles that are greater than 90° with adjacent ones of the reflective panels 34. In so arranging the reflective panels 34, the light emitted from the bulbs 48 can be reflected toward a center of the chamber 32 and/or may provide a greater degree of once-reflected light contacting the article 40 when compared to right angle or acute angle joining of reflective panels 34. The term once-reflected light can be utilized herein to refer to light that has contacted only one of the reflective panels 34 prior to contacting the article 40. It may be beneficial to increase a proportion of once-reflected light contacting the article 40 as it is possible for an intensity of the light to decrease from an incident power after undergoing reflection.

Referring further to FIGS. 6-13, the shelf 36 can be coupled to the rear panel 188 and may be adjustable in a vertical direction such that the shelf 36 is suspended above the bottom panel 176. More specifically, the shelf 36 can be provided with a protrusion 200 that can be received within an aperture 204 that is defined by the rear panel 188. In the depicted example, the shelf 36 is shown as a wire-frame shelf. The wire-frame structure of the shelf 36 is transparent to the wavelength of the light emitted from the bulbs 48 in that gaps or voids exist between individual wires of the wire-frame structure. Accordingly, similar to the above described example, light emitted from the bulbs 48 can contact and ultimately disinfect or cleanse a surface of the article 40 that is in direct contact with the shelf 36. It is contemplated that the material from which the wire-frame structure of the shelf 36 is made may provide a degree of reflection of the wavelength of light that is emitted from the bulbs 48 such that the light is additionally reflected. For example, the wire-frame structure of the shelf 36 may be made from a polished aluminum material. Alternatively, it is contemplated that a coating may be applied to the shelf 36 that provides a degree of reflection of the wavelength of light that is emitted from the bulbs 48 such that the light is additionally reflected. For example, the wire-frame structure may be coated with an aluminum coating. In some examples, the lock assembly 60 may be provided with one of the reflective panels 34 on one or more interior surfaces of the lock assembly 60 that are exposed to the light emitted from the bulbs 48. In such an example, the reflective panel(s) 34 that is coupled to the lock assembly 60 may be referred to as a lock panel 208.

Referring still further to FIGS. 6-13, the lock assembly 60 can include a locking tab 212 that is coupled to a release handle 216. Actuation of the release handle 216 by a user can operate the locking tab 212 between an extended position and a retracted position. When the locking tab 212 is in the extended position and the access door 28 is in a closed position, the locking tab 212 can engage with a tab receiver 220 that defines a tab aperture 224. The tab aperture 224 receives the locking tab 212 to retain the access door 28 in the closed position. During a cleansing cycle, the lock assembly 60 can decouple the release handle 216 from the locking tab 212 to prevent a user from accessing the chamber 32 while the bulbs 48 are in their energized state. Alternatively, the lock assembly 60 can impede actuation of the release handle 216 to prevent the user from accessing the chamber 32 while the bulbs 48 are in their energized state.

Referring more specifically to FIGS. 6-9, the sanitation device 20 can be provided with feet 228. In some examples, the feet 228 may be movable in a vertical direction relative to the housing 24 such that a position of the bottom wall 152 relative to a surface upon which the sanitation device 20 rests may be adjusted. For example, adjusting the feet 228 can be utilized to adjust a degree of clearance between the bottom wall 152 of the housing 24 and the surface upon which the sanitation device 20 rests or the feet 228 may be adjusted to level the sanitation device 20. In the depicted example, the bulb assemblies 44 have a length that substantially corresponds with a height of the left panel 180, the right panel 184, the rear panel 188, and/or the front panel 192. The bulbs 48 of the bulb assemblies 44 may be responsible for a substantial portion of a length of the bulb assemblies 44. Accordingly, a majority of the height of the chamber 32 may be directly exposed to the light emitted by the bulbs 48 prior to reflection off of one of the reflective panels 34. A power cord 236 is coupled to the sanitation device 20 and configured to receive power from a power source and deliver the received power to various electrical components of the sanitation device 20 (e.g., the bulb assemblies 44, the lock assembly 60, the user interface 64, the display screen 68, the button 72, the controller 76, etc.). Various electrical components and/or operating components can be housed within the cavity 26 that exists between the housing 24 and the reflective panels 34. For example, the components responsible for operating the sanitation device 20 may be housed in the cavity 26 that exists between the bottom wall 152 of the housing 24 and the bottom panel 176 of the chamber 32. In so positioning the operating components, heat that can be generated during the operation of the sanitation device 20 may be less likely to impact the operating components. In some examples, the sanitation device 20 of the present example may be provided with the circulation device 128 described above. For example, the circulation device 128 may be positioned between the top wall 148 and the top panel 172 with one or more of the vents 132 provided in the top wall 148 and the orifice 130 provided in the top panel 172. In examples that employ the circulation device 128, active cooling may be provided to the sanitation device 20 to decrease a temperature of the sanitation device 20 and/or improve an operating efficiency of the sanitation device 20 by actively cooling the sanitation device 20. Alternatively, the circulation device 128 may be omitted and passive cooling of the sanitation device 20 may be relied upon. That is, natural convection or heat flow from warmer regions to cooler regions may be relied on to provide the passive cooling of the sanitation device 20.

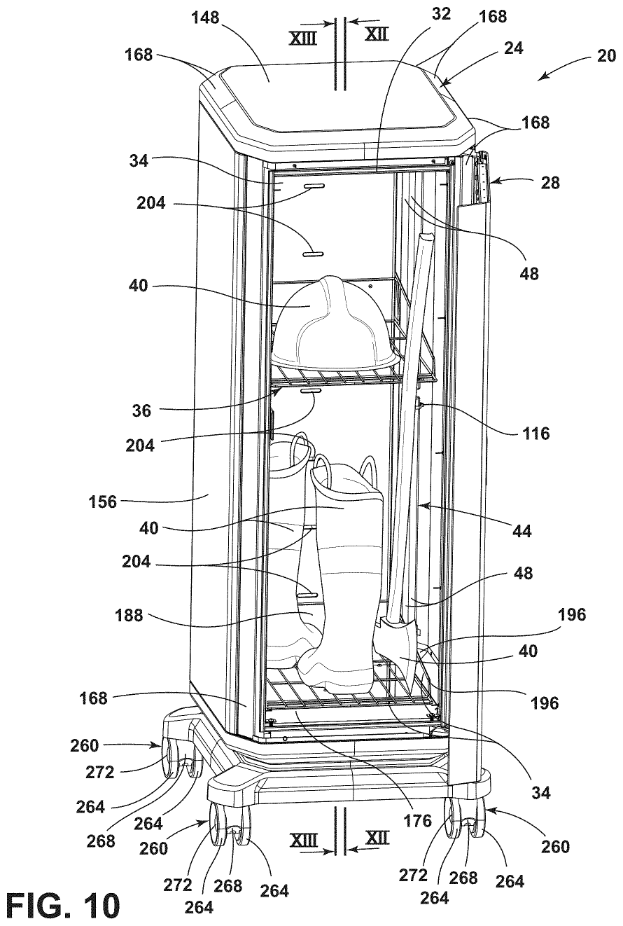

With specific reference to FIGS. 10-13, as with the above example, the sanitation device 20 includes the power cord 236 coupled thereto. In the depicted example, the sanitation device 20 is larger in at least the height or vertical direction when compared to the example shown in FIGS. 6-9. Accordingly, a plurality of the bulb assemblies 44 can be provided on a given one of the junction panels 196 that are coupled to adjacent ones of the reflective panels 34 that extend along the vertical direction (e.g., the left panel 180, the right panel 184, the rear panel 188, and/or the front panel 192). The plurality of bulb assemblies 44 that are arranged on a given one of the junction panels 196 can be oriented such that the bulbs 48 of the individual bulb assemblies 44 are adjacent to one another while the receptacles 52 are arranged proximate to the top panel 172 and the bottom panel 176. In so arranging the bulb assemblies 44, a greater percentage of the volume of the chamber 32 may be provided with light that is directly emitted from the bulbs 48 prior to reflection of the light emitted by the bulbs 48. As with the above example, the bulb assemblies 44 may extend along a majority of the height of the chamber 32. Accordingly, a combined length of the plurality of bulb assemblies 44 at a given one of the junction panels 196 can substantially correspond with the height dimension of the given junction panel 196. In some examples, the sanitation device 20 of the present example may be provided with the circulation device 128 described above. For example, the circulation device 128 may be positioned between the top wall 148 and the top panel 172 with one or more of the vents 132 provided in the top wall 148 and the orifice 130 provided in the top panel 172. In examples that employ the circulation device 128, active cooling may be provided to the sanitation device 20 to decrease a temperature of the sanitation device 20 and/or improve an operating efficiency of the sanitation device 20 by actively cooling the sanitation device 20. Alternatively, the circulation device 128 may be omitted and passive cooling of the sanitation device 20 may be relied upon. That is, natural convection or heat flow from warmer regions to cooler regions may be relied on to provide the passive cooling of the sanitation device 20.

Referring again to FIGS. 10-13, the sanitation device 20 may be provided with a cord-keeping structure 240 coupled thereto. The cord-keeping structure 240 may be coupled to the rear wall 164 of the housing 24. The cord-keeping structure 240 includes an upper portion 244 and lower portion 248. The upper portion 244 and the lower portion 248 of the cord-keeping structure 240 can be utilized to store at least a portion of the power cord 236 when the sanitation device 20 is not in use, when the sanitation device 20 is being transported, or when an entirety of the length of the power cord 236 is not required to reach a power source (e.g., a wall outlet). In the depicted example, one or more of the chamfered corners 168 that are positioned on either side of the rear wall 164 may be provided with a grab handle 252. The grab handle(s) 252 can be utilized by a user in transporting the sanitation device 20 from a first location to a second location. To aid in the positioning or transport of the sanitation device 20 the sanitation device 20 of the depicted example can be provided with a support base 256. The support base 256 is configured to receive the sanitation device 20 on an upper surface thereof such that the support base 256 engages with the bottom wall 152 of the housing 24. To aid in movement of the sanitation device 20 along a horizontal plane, the support base 256 is provided with roller assemblies 260. The roller assemblies 260 each include one or more wheels 264 that are coupled to a support peg 268 by an axle 272. The support pegs 268 can be coupled to an underside of the support base 256 in a manner that permits rotation about a vertical axis (e.g., caster). The vertical axis of rotation for the support peg 268 can be defined by the engagement between the support peg 268 and the support base 256. In the depicted example, one of the roller assemblies 260 is provided at each corner of the sanitation device 20 and corresponds with one of the chamfered corners 168. The roller assemblies 260 can be provided with a step lock 276 that is coupled to the support peg 268. Placing the step lock 276 in a locked position can prevent rotational motion of the wheels 264 about the axles 272 and retain the sanitation device 20 in a given location.

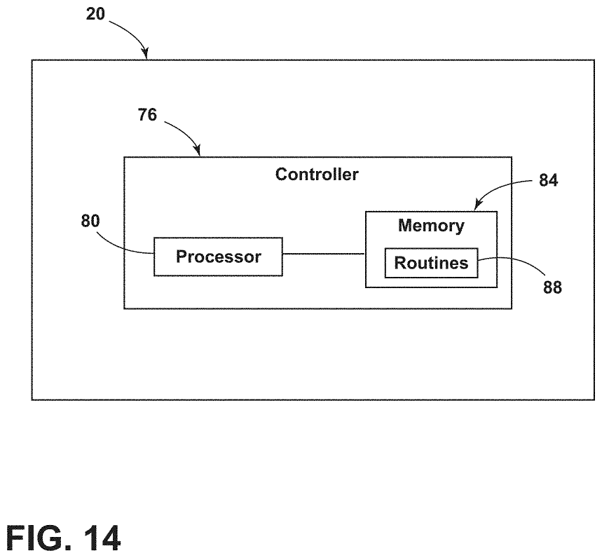

Referring to FIG. 14, the sanitation device 20 includes the controller 76. The controller 76 includes the processor 80 and the memory 84. The memory 84 stores software routines 88 that are executable by the processor 80. In various situations, the software routines 88 within the memory 84 may be accessed by the controller 76 and/or the processor 80 in response to an input from the sanitation device 20 (e.g., the lock assembly 60, the user interface 64, a thermostat, a temperature sensor, etc.). For example, in response to an input from the user interface 64, the processor 80 may access the software routines 88 stored within the memory 84 to initiate a cleansing cycle. In examples that employ a temperature sensor, the software routines 88 can reference the temperature sensed by the temperature sensor prior to initiating the cleansing cycle. The temperature sensor may be an active temperature sensor that senses a current temperature within the cavity 26 and/or the chamber 32. For example, the active temperature sensor may be a thermocouple that quantitatively senses the current temperature. Alternatively, the temperature sensor may be a passive temperature sensor that senses a current temperature within the cavity 26 and/or the chamber 32 in a binary fashion. For example, the passive temperature sensor may be a thermal fuse that prevents progression of the cleansing cycle when the sensed temperature exceeds the predetermined threshold. In such an example, once the temperature within the cavity 26 and/or the chamber 32 has fallen below the predetermined threshold, the thermal fuse may permit progression of the cleansing cycle (e.g., initiation of the cleansing cycle). The thermal fuse may be a multi-use thermal fuse or a one-time use thermal fuse. The passive temperature sensor may be referred to as a qualitative temperature sensor. Regardless of whether the temperature sensor is passive or active, the temperature sensor can be utilized as an input to the controller 76 that can impact whether or not power is supplied to the bulb(s) 48. By maintaining a temperature within the sanitation device 20 below the predetermined threshold, an operation efficiency of the bulb(s) 48 may be maintained. Initiation of the cleansing cycle can include determining that the access door 28 is closed, determining that the lock assembly 60 is in the engaged state, and energizing the bulb(s) 48. In some examples, the cleansing cycle can include determining that the access door 28 is closed and energizing the bulb(s) 48. In such examples, the lock assembly 60 may be omitted from the sanitation device 20 or the lock assembly 60 may not be employed during the cleansing cycle. Rather, in such examples, in the event that the access door 28 is moved from the closed position (e.g., toward the open position) during the cleansing cycle, then the controller 76 may place the bulb(s) 48 in the de-energized state in response to the access door 28 no longer being in the closed position.

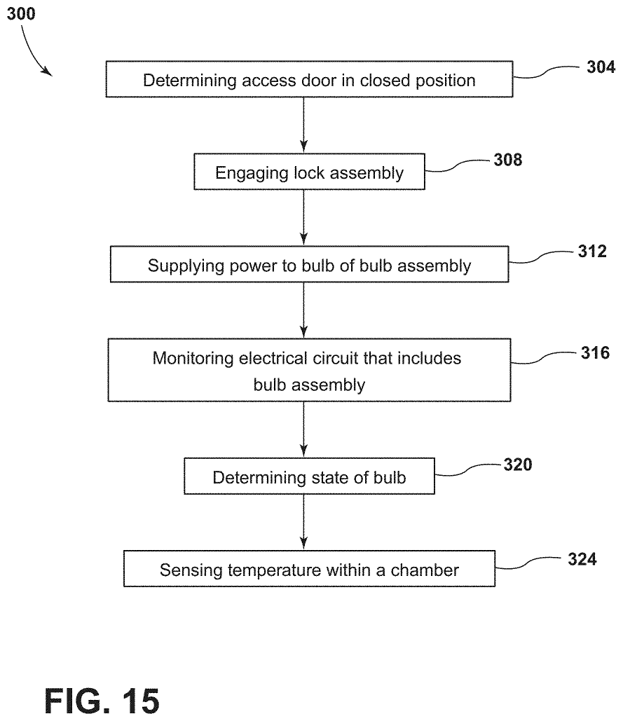

Referring now to FIG. 15, a method 300 of operating the sanitation device 20 is shown. The method 300 includes step 304 of determining that the access door 28 is in the closed position. When the method 300 has determined that the access door 28 is in the closed position at step 304, the method 300 may advance to step 308 of engaging the lock assembly 60. In some examples, the step 308 of engaging the lock assembly 60 may be initiated by an input from the user by way of the user interface 64. For example, after determining that the access door 28 is in the closed position at step 304, the lock assembly 60 may remain in the disengaged state until such time as the controller registers that the button 72 has been actuated. In such an example, the actuation of the button 72 can initiate a cleansing cycle of the sanitation device 20. After a determination that the access door 28 is in the closed position at step 304 and the engagement of the lock assembly 60 at step 308, the method 300 may advance to step 312 of supplying power to the bulb(s) 48 of the bulb assembly 44 or bulb assemblies 44.

Referring again to FIG. 15, the method 300 also includes step 316 of monitoring an electrical circuit that includes the bulb assembly 44 or bulb assemblies 44. By monitoring the electrical circuit at step 316, the method 300 may determine a state (e.g., energized vs. de-energized) of the bulb(s) 48 of the bulb assembly 44 or bulb assemblies 44 at step 320. In the event that the monitoring of the electrical circuit at step 316 results in a determination at step 320 that at least one bulb 48 is in the de-energized state when the energized state is intended, then the controller 76 may communicate an error message to the user by way of the display screen 68 and/or the perimeter structure 74. In so doing, under-dosing of the article 40 during the cleansing cycle can be mitigated. As used herein, the term under-dosing is intended to refer to a decrease in the intended intensity of light exposure delivered from the bulb(s) 48 and/or a decrease in the intended duration of light exposure delivered from the bulb(s) 48. A decrease in the intended intensity of light exposure delivered from the bulb(s) 48 can be caused by one or more of the bulbs 48 failing or “going out.” A decrease in the intended duration of light exposure delivered from the bulb(s) 48 can be caused by the user cancelling or exiting the cleansing cycle prior to completion of the cleansing cycle. In various examples, the sanitizing device 20 can include a temperature sensor that is utilized to sense a temperature within the chamber 32 at step 324. In one specific example, the temperature sensor may be a thermal fuse that halts or prevents the progression of the cleansing cycle when the temperature within the chamber 32 and/or the cavity 26 exceeds a predetermined threshold. If the sensed temperature exceeds the predetermined threshold, then the sanitation device 20 can initiate a cooling cycle. In examples that employ the circulation device 128, the circulation device 128 may be activated during the cleansing cycle such that the cooling cycle and the cleansing cycle are performed simultaneously. Additionally, or alternatively, the circulation device 128 may be activated at the conclusion of the cleansing cycle such that the cooling cycle and the cleansing cycle are performed in a sequential manner. In examples that do not employ the circulation device 128, the cooling cycle can be initiated as a result of a given number, N, cleansing cycles being completed in a first predetermined amount of time, X. In such an example, the cooling cycle may be accomplished with passive air flow or convection for a second predetermined period of time, Y. It may be beneficial to maintain the temperature within the chamber 32 below the predetermined threshold to maintain an efficiency of the cleansing cycle.

The cleansing cycle may vary based upon the volume of the chamber 32 and/or the number of bulb assemblies 44 employed. For example, the exemplary sanitation device 20 depicted in FIGS. 1-5 is provided with two bulb assemblies 44 and may have a cleansing cycle duration of less than one minute (e.g., 30 seconds). The exemplary sanitation device 20 depicted in FIGS. 6-9 is provided with four bulb assemblies 44 and may have a cleansing cycle duration of two minutes. The exemplary sanitation device 20 depicted in FIGS. 10-13 is provided with eight bulb assemblies 44 and may also have a cleansing cycle duration of two minutes despite the larger volume of the chamber 32. In testing, the exemplary sanitation device 20 depicted in FIGS. 1-5 achieved kill rates of Escherichia coli (E. coli) and Methicillin-resistant Staphylococcus aureus (MRSA) of up to 99.9999% and a kill rate of Clostridium difficile (C. diff) of up to 99.9990%. In testing, the exemplary sanitation device 20 depicted in FIGS. 6-9 achieved kill rates of E. coli and MRSA of up to 99.9999% and a kill rate of C. diff of up to 99.9930%. In testing, the exemplary sanitation device 20 depicted in FIGS. 10-13 achieved kill rates of E. coli and MRSA of up to 99.9999% and a kill rate of C. diff of up to 99.9987%.

Modifications of the disclosure will occur to those skilled in the art and to those who make or use the concepts disclosed herein. Therefore, it is understood that the embodiments shown in the drawings and described above are merely for illustrative purposes and not intended to limit the scope of the disclosure, which is defined by the following claims as interpreted according to the principles of patent law, including the doctrine of equivalents.

It will be understood by one having ordinary skill in the art that construction of the described concepts, and other components, is not limited to any specific material. Other exemplary embodiments of the concepts disclosed herein may be formed from a wide variety of materials, unless described otherwise herein.

For purposes of this disclosure, the term “coupled” (in all of its forms: couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature, or may be removable or releasable in nature, unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, and the nature or numeral of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes, or steps within described processes, may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present disclosure, and further, it is to be understood that such concepts are intended to be covered by the following claims, unless these claims, by their language, expressly state otherwise.

What is claimed is:

1. A sanitation device, comprising:
a housing;
an access door coupled to the housing and movable between an open position and a closed position, wherein the access door comprises an interior surface;
a plurality of reflective panels positioned within the housing, wherein one of the plurality of reflective panels is coupled to the interior surface of the access door;
a cavity defined by the housing and at least one of the plurality of reflective panels;
a chamber that is defined by the plurality of reflective panels;
a shelf positioned within the chamber and configured to receive an article; and
one or more bulb assemblies positioned within the chamber, wherein the one or more bulb assemblies each comprises a bulb and a receptacle, wherein the bulb is operable between an energized state and a de-energized state, wherein the bulb emits light with a wavelength in the range of about 100 nm to about 400 nm when in the energized state, and wherein each of the reflective panels are configured to reflect the light emitted by the bulb.

2. The sanitation device of claim 1, wherein the access door comprises a rearward edge, and wherein the rearward edge is positioned within the cavity when the access door is in the open position.

3. The sanitation device of claim 1, wherein the chamber is generally cylindrical in shape.

4. The sanitation device of claim 3, wherein the one or more bulb assemblies comprises an upper bulb assembly and a lower bulb assembly, and wherein the upper bulb assembly is diametrically opposed to the lower bulb assembly.

5. The sanitation device of claim 4, wherein the shelf is suspended between the upper bulb assembly and the lower bulb assembly.

6. The sanitation device of claim 1, further comprising:
a circulation device, wherein the circulation device is positioned within the cavity.

7. The sanitation device of claim 1, wherein the housing comprises:
a top wall;
a bottom wall;
a left wall;
a right wall; and
a rear wall, wherein adjacent edges of the top wall, the bottom wall, the left wall, the right wall, and the rear wall are coupled to one another by chamfered corners.

8. The sanitation device of claim 7, wherein one or more of the chamfered corners that positioned on either side of the rear wall is provided with a grab handle.

9. The sanitation device of claim 7, wherein the plurality of reflective panels comprises:
a top panel;
a bottom panel;
a left panel;
a right panel;
a rear panel;
a front panel; and
a junction panel, wherein the junction panel corresponds with, and is parallel to, at least one of the chamfered corners of the housing, and wherein the junction panel couples adjacent edges of two panels chosen from the top panel, the bottom panel, the left panel, the right panel, the rear panel, and the front panel.

10. The sanitation device of claim 9, wherein the junction panel forms an obtuse angle with the adjacent edges of the two panels chosen from the top panel, the bottom panel, the left panel, the right panel, the rear panel, and the front panel.

11. The sanitation device of claim 9, wherein at least one of the one or more bulb assemblies is arranged on the junction panel.

12. The sanitation device of claim 1, further comprising:
a temperature sensor that senses a temperature within at least one location chosen from the cavity and the chamber.

13. The sanitation device of claim 1, further comprising:
a lock assembly that is operable between an engaged state and a disengaged state, wherein the lock assembly prevents the access door from moving from the closed position to the open position when the lock assembly is in the engaged state.

14. The sanitation device of claim 13, wherein the lock assembly is placed in the engaged state during a cleansing cycle.

15. The sanitation device of claim 1, further comprising:
a user interface, wherein the user interface comprises a display screen and a button.

16. The sanitation device of claim 1, further comprising:
a controller having a processor and memory, wherein the memory comprises executable software routines stored therein.

17. A method of operating a sanitation device, the method comprising the steps of:
determining an access door is in a closed position;
supplying power to a bulb of a bulb assembly;
monitoring an electrical circuit that comprises the bulb assembly;
determining whether the bulb of the bulb assembly is in an energized state or a de-energized state based upon the monitoring of the electrical circuit that comprises the bulb assembly; and
sensing a temperature within a chamber with a temperature sensor.

18. The method of operating a sanitation device of claim 17, further comprising:
communicating an error message to a user if the bulb of the bulb assembly is determined to be in the de-energized state during a cleansing cycle.

19. The method of operating a sanitation device of claim 17, further comprising:
terminating a cleansing cycle if the temperature sensed by the temperature sensor exceeds a predetermined threshold.

20. The method of operating a sanitation device of claim 19, wherein the temperature sensor is a thermal fuse.
